(19) United States
(12) Patent Application Publication
(10) Pub. No.: US 2021/0295950 A1
(43) Pub. Date: Sep. 23, 2021

(54) METHOD FOR ENCODING AND DECODING OF QUALITY VALUES OF A DATA STRUCTURE

(71) Applicants: Gottfried Wilhelm Leibniz Universität Hannover, Hannover (DE); Stanford University, Stanford, CA (US)

(72) Inventors: Jan VOGES, Hannover (DE); Mikel HERNAEZ, Stanford, CA (US); Jörn OSTERMANN, Hannover (DE)

(21) Appl. No.: 16/341,307

(22) PCT Filed: Oct. 12, 2016
(86) PCT No.: PCT/EP2016/074442

§ 371 Date: Apr. 11, 2019

(51) Int. Cl. G16B 30/10; H03M 7/30; G06F 16/215; G06F 16/21; G06F 16/22

(52) U.S. Cl. CPC G16B 30/10; H03M 7/3059; G06F 16/2237; G06F 16/217; G06F 16/215

(57) ABSTRACT

The invention relates to a method for encoding of quality values of a data structure, whereby said data structure includes a plurality of continuous fragments, each continuous fragment comprises a sequence of symbols derived from a symbol alphabet and corresponds to a segment of one reference sequence of one or more reference sequences, whereby each continuous fragment is aligned to locus indexes of one of said reference sequence and at least a portion of said continuous fragments overlap at an aligned locus index, and includes, further, a plurality of quality values, each quality value is derived from a quality value alphabet and is assigned to a corresponding symbol of one of the continuous fragments, whereby each quality value indicates a likelihood that the corresponding symbol in the corresponding continuous fragment is correct, wherein the method comprises the steps executable by a data processing system: —determine the quality values at a specific locus index, which are assigned to symbols of continuous fragments aligned to said specific locus index; —calculate an estimation certainty at the specific locus index based on the determined quality values, whereby said estimation certainty indicates a likelihood of correctness for each quality value of the determined quality values in relation to the corresponding symbols; and —encode the determined quality values by transform of each determined quality values into a transformed quality value based on the calculated estimation certainty.

The present invention relates to a method and to a corresponding device for encoding of quality values of a data structure, especially of genomic data stored as such data structure. The present invention relates also to a method for decoding of quality values of a data structure, which was encoded by a method of the present invention.

Due to novel high-throughput sequencing (HTS) and/or next-generation sequencing (NGS) technologies, the sequencing of huge amounts of genetic information has become affordable. Because of this float of data, IT costs may count a major obstacle compared to sequencing costs. High-performance compression of genomic data is required to reduce to storage size and transmission costs.

Sequencing machines produce a multitude of readouts (reads in short) of fragments of e.g. DNA material. During the sequencing process, a quality value, also known as quality score, is assigned to each nucleotide in a readout. These quality values express the confidence that the corresponding nucleotide has been a readout correctly. The readouts (e.g. the sequence of nucleotides together with the associated quality values) and the associated read identifiers are commonly stored in the FASTQ format.

In Peter J A Cock, Christopher J Fields, Naohisa Goto, Michael L Heuer, and Peter M Rice. The Sanger FASTQ format for sequences with quality scores, and the Solexa/Illumina FASTQ variants. Nucleic Acids Research, 38(6):1767-1771, 2010, is a FASTQ file format for sequences with quality scores disclosed.

After the raw data has been generated, some of the most common subsequent processing steps are:
a) Reference-based alignment of the reads with tools such BWA (Heng Li and Richard Durbin. Fast and accurate short read alignment with Burrows-Wheeler transform. Bioinformatics, 25(14):1754-1760, 2009), Bowtie (Ben Langmead and Steven L Salzberg. Fast gapped-read alignment with Bowtie
2. Nature Methods, 9(4):357-359, 2012; Ben Langmead, Cole Trapnell, Mihai Pop, and Steven L Salzberg. Ultrafast and memory-efficient alignment of short DNA sequences to the human genome. Genome Biology, 10(3):R25.1-10, 2009), mrsFAST (Faraz Hach, Fereydoun Hormozdiari, Can Alkan, Farhad Hormozdiari, Inanc Birol, Evan E Eichler, and S Cenk Sahinalp. mrsFAST: a cache-oblivious algorithm for short-read mapping. Nature Methods, 7(8):576-577, 2010) or GEM (Santiago Marco-Sola, Michael Sammeth, Roderic Guigo, and Paolo Ribeca. The GEM mapper: fast, accurate and versatile alignment by filtration. Nature Methods, 9(12):1185-1188, 2012) or
b) De-novo assembly of the reads with tools such ABySS (Jared T Simpson, Kim Wong, Shaun D Jackman, Jacqueline E Schein, Steven J M Jones, and Inanc Birol. ABySS: a parallel assembler for short read sequence data. Genome Research, 19(6):1117-1123, 2009) or SPAdes (Anton Bankevich, Sergey Nurk, Dmitry Antipov, Alexey A Gurevich, Mikhail Dvorkin, Alexander S Kulikov, Valery M Lesin, Sergey I Nikolenko, Son Pham, Andrey D Prjibelski, Alexey V Pyshkin, Alexander V Sirotkin, Nikolay Vyahhi, Glenn Tesler, Max A Alekseyev, and Pavel A Pevzner. SPAdes: A New Genome Assembly Algorithm and Its Applications to Single-Cell Sequencing. Journal of Computational Biology, 19(5):455-477, 2012).

During the alignment or assembly process, additional information is generated for each read, such as the mapping positions or the CIGAR strings. The later express of different operations needed to be performed on a read so that it maps perfectly to the reference used for alignment or assembly. The reads are extended with this additional information to form so-called alignments, which are usually stored in this SAM format (Heng Li, Bob Handsaker, Alec Wysoker, Tim Fennell, Jue Ruan, Nils Homer, Gabor Marth, Goncalo Abecasis, and Richard Durbin. The Sequence Alignment/Map format and SAMtools. Bioinformatics, 25(16):2078-2079, 2009; January Voges, Marco Munderloh, and Jön Ostermann. Predictive Coding of Aligned Next-Generation Sequencing Data. In Data Compression Conference (DCC), pages 241-250, Snowbird, UT (US), 2016. IEEE).

A donor sequence of a donor genome s=(S₁, . . . s_{ó}, . . . , S_{Ls}) is a sequence of length Lₛ, where the symbols s_{ó}come from an alphabet S with cardinality |S|. A continuous fragment f, of length L_{f}, read out by a sequencing method from a donor sequence s can be written as f=(f₁, f_{ø}, . . . , f_{Lf}). A quality value q_{ø} might be associated with a symbol f_{ø}. Usually, there are corresponding quality values q_{ø} for all symbols f_{ø}. Furthermore, since the sequencing process might be error-prone, the fragments may contain insertions and/or deletions of arbitrary size (with respect to the corresponding donor sequence s), or an arbitrary number of subsequent symbols that have not been read out correctly. Single symbol errors are in a biological context also known as single nucleotide polymorphisms (SNPs). Generally, multiple fragments fᵢ might overlap due to redundant reading of the donor sequence s by the employed sequencing method. The number of times a specific locus in a donor sequence s has been read out is called sequencing depth N. The average sequencing depth over all loci is called coverage. Furthermore, multiple (so-called homologous) donor sequences sₕ containing symbols from the same alphabet S may exist or be assumed, whereby a specific fragment usually has been read out from one specific donor sequence. These sequences are in principle similar, but may contain arbitrary alterations. The total number of homologous donor sequences is referred to as the polyploidy h. If at a specific locus I all homologous donor sequences contain the same symbol, the donor sequences are denoted as being homozygous at that specific locus; otherwise, the donor sequences are denoted as being heterozygous at that specific locus. A specific symbol at a locus I on one donor sequence is called allele. Finally, a specific combination of symbols (i.e. alleles) across all h donor sequences at a locus I is called the genotype at locus.

It is an aspect of the present invention to provide a better encoding and compression method for compressing mapped and/or aligned genomic data or similar data structures. It is a further aspect of the present invention to provide a decoding or decompression method for decoding such encoded genomic data.

According to claim 1, a method for encoding of quality values of a data structure is proposed. The data structure may include at least one reference sequence, whereby each reference sequence comprises a sequence of symbols derived from a symbol alphabet. Such reference sequence could be a donor genome including a plurality of nucleotide symbols from a nucleotide symbol alphabet (usually A, C, G, T). In the broadest sense, one or more reference sequences are assumed, which underlies the present invention.

The data structure includes a plurality of continuous fragments, whereby each continuous fragment comprises a sequence of symbols derived from a symbol alphabet, the same symbol alphabet according to the reference sequences. A continuous fragment corresponds to a segment of one reference sequence of one or more reference sequences, whereby each continuous fragment is aligned to locus indexes of one of said reference sequence. The locus index means the position of a symbol within a (assumed) reference sequence or within an aligned continuous fragment. The alignment means that the symbols of the continuous fragments correspond with the correct (or assumed) position in the reference sequence.

A continuous fragment as a sequence of symbols may include insertions of one or more symbols, deletions of one or more symbols and/or alterations of one or more symbols, especially in view of one of said reference sequence.

At least a portion of the continuous fragments overlap at an aligned locus index, so that at a given locus index a plurality of symbols of different continuous fragments exists.

The continuous fragments could be produced by a DNA sequencing machine (e.g. as a readout).

Furthermore, the data structure includes a plurality of quality values, whereby each quality value is derived from a quality value alphabet. Each quality value is assigned to a corresponding symbol of one of the continuous fragments and indicates a likelihood that the corresponding symbol in the corresponding fragment is correct, for example correct in view of one of said reference sequence. However, it could be that the reference sequence is also incorrect, so that the quality value also indicates a likelihood that the corresponding symbol in the corresponding fragment is correct in view of its producing method (e.g. DNA sequencing).

The quality values could be quality scores produced by a DNA sequencing machine.

For example, such a data structure is saved in a data file, for example a SAM-file.

The method for encoding such quality values to reduce the information density for reducing the memory space of the data structure, comprises the following steps executable by a data processing system. At first, at a specific locus index, quality values at said specific locus index are determined. These quality values are assigned to symbols of continuous fragments, which are aligned to the specific locus index. In other words, all quality values, which are assigned to symbols of continuous fragments at the specific locus index, are determined. The maximum number of quality values is the maximum number of overlapping continuous fragments at the specific locus index.

In the next step, an estimation certainty at the specific locus index is calculated. The estimation certainty is calculated based on the determined quality values at the specific locus index, whereby the estimation certainty indicates a likelihood of correctness of each quality value of the determined quality value in relation to the corresponding symbols.

For example, if at a specific locus index in a first continuous fragment and in a second continuous fragment the same symbol but different quality values are found, it is not sure, that the symbol in the continuous fragments at the specific locus index is correct. Because one symbol has a high quality value and the second, identical symbol has a low quality value, the quality value of the first symbol or the quality value of the second symbol could be correct. Therefore, the likelihood of correctness of the quality values in relation to the corresponding symbol is in the median of both quality values.

Based on this calculated estimation certainty at the specific locus index, each determined quality value is encoded by transforming each determined quality value into a transformed quality value. Based on the fact, that the estimation certainty at the specific locus index indicates a likelihood of correctness of each quality value in relation to the corresponding symbol, each quality value could transform in a transformed quality value for encoding the quality values. The transformed quality values have a lower information density so that e.g. the memory space of the data file including this data structure can be reduced. For example, to reduce the information density of the transformed quality values, only a part of the quality value alphabet can used for the transformed quality values.

To encode all quality values of the data structure, these preceding method steps are performed for each locus index of the reference sequence.

The data structure may provide in one or more data files. If all quality values are transformed, the data structure may be re-saved in one or more data files, e.g. in the same data files.

Therefore, the method for encoding of quality values by transform the quality values into transformed quality values is, in the broadest sense, a method for compression of quality values, because the information density is reduced.

In a first variant, the estimation certainty is calculated in form of a quality value derived from the quality value alphabet, whereby the determined quality values are transformed by setting each quality value to the estimation certainty if the estimation certainty is greater or equal than the quality value to be transformed.

The transformed quality values are compressed using a compression algorithm. Based on the reducing of the information density of the transformed quality values, the compression using a compression algorithm is more effective and the needed memory space could more reduced.

In a second variant, a quantization characteristic is selected based on the estimation certainty at the specific locus index. A quantization characteristic associates all quality values of the quality value alphabet to one or more quantized quality values, whereby usually the number available quantized quality values are lower than the total number of available quality values from the quality value alphabet. The determined quality values are transformed by quantizing each determined quality value into a quantized quality value based on said selected quantization characteristic, whereby the estimation certainty or the selected quantization characteristic is assigned to the specific locus index and the quantized quality value is used as a transformed quality value.

In an embodiment, the quantization characteristic is selected based on the estimation certainty in such a way that the determined quality values at a first locus index with the first estimation certainty are quantized more coarsely than the determined quality values at a second locus index with a second estimation certainty, if the first estimation certainty is higher than the second estimation certainty.

In other words, if the estimation certainty is very high, the likelihood of correctness of the quality values in relation to the corresponding symbols (or the corresponding symbol itself) is very high also. In this case, the quantization characteristic can be coarse. Coarse means, that a total number of available quantized quality values is very low (e.g. 2 or 3). However, if the estimation certainty is low and therefore the likelihood of correctness of the quality values in relation to the corresponding symbols (or the corresponding symbol itself) is very low also, a quantization characteristic is subtler so that the total number of available quantized quality values is much more higher (e.g. 5 or greater).

In short, if the likelihood of correctness of the quality values in relation to the corrsponding symbols is very low, the quantization is subtler. If the likelihood of correctness is higher, the quantization is coarser.

If a first estimation certainty is higher than a second estimation certainty, a first quantization characteristic based on the first estimation certainty has less available quantized quality values than a second quantization characteristic based on the second estimation certainty.

Based on this method, it is possible to reduce in average a normal quality value using 8 bits to less than 1 bit.

In a further embodiment, a step size of the quantization characteristic is selected based on the estimation certainty.

In a further embodiment, an entropy-coding step of the quantized quality value is controlled by using the estimation certainty.

In the case that the data structure comprises more than one reference sequence and the continuous fragments corresponds to a segment in the sequence of one of the reference sequence, all possible symbol combinations are determined based on the number of reference sequences. For example, if the reference sequence is a human genome, the data structure comprises two reference sequences. Each continuous fragment could correspond to a sequence in the first reference sequence or the second reference sequence, whereby this assignment, which continues fragments, correspond to the first or second reference sequence, is unknown. Based on the four available nucleotide symbols, ten possible combinations could be identified at a specific locus index for the 2 reference sequences.

For each symbol combination, a likelihood of occurrence is calculated based on the determined quality values. The estimation certainty at the specific locus index is calculated based on the likelihood of occurrence of each symbol combination.

According to claim 12, a method for decoding of transformed quality values is proposed. The transformed quality values are encoded by a method of encoding of quality values according to one of the claims 4 to 11, which uses a quantization characteristic. Initially, the transformed quality values at a specific locus index are determined. Furthermore, the estimation certainty or the selected quantization characteristic assigned to said specific locus index is also determined. If the estimation certainty is assigned to the specific locus index, based on this estimation certainty at the specific locus index is used to select a quantization characteristic. Otherwise, the assigned quantization characteristic is used.

Based on a determined quantization characteristic, each quantized quality value as a transformed quality value is retransformed into a re-quantized quality value. In relation to the total number of quality values in the quality value alphabet, the requantized quality values are more coarse. A continuous part of quality values in the quality value alphabet are projected to one re-quantized quality value. Therefore, the method for encoding and decoding realizes a lossy compression.

Therefore, the method for decoding of quality values by retransform the quantized quality values into re-quantized quality values is, in the broadest sense, a method for decompression of compressed quality values.

The present invention is described in more detail by reference to the following figures:

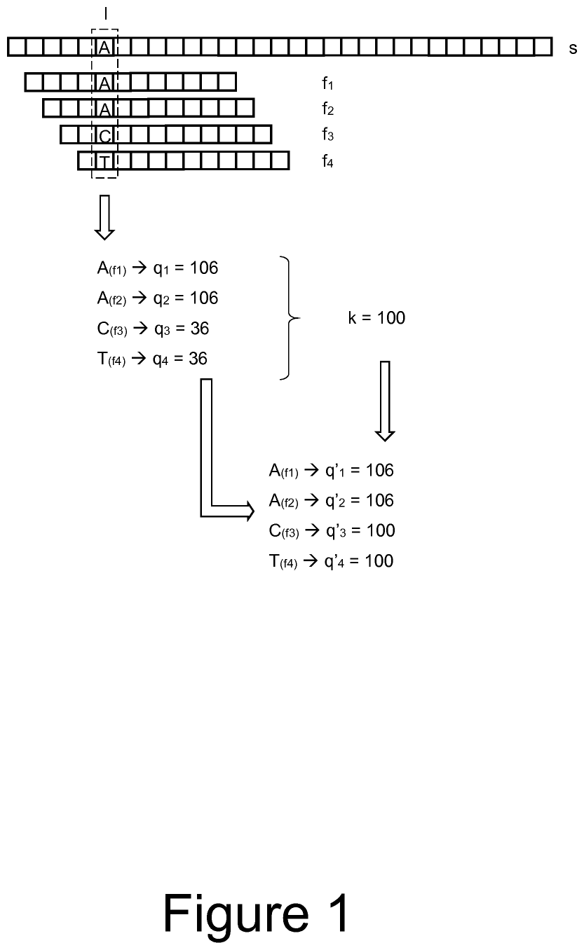

FIG. 1 description of a first embodiment;

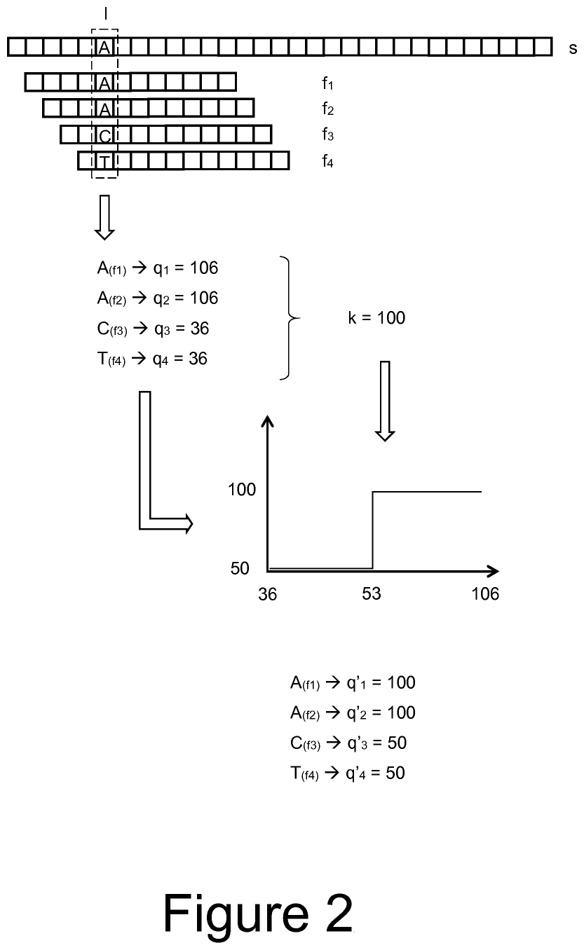

FIG. 2 description of a second embodiment;

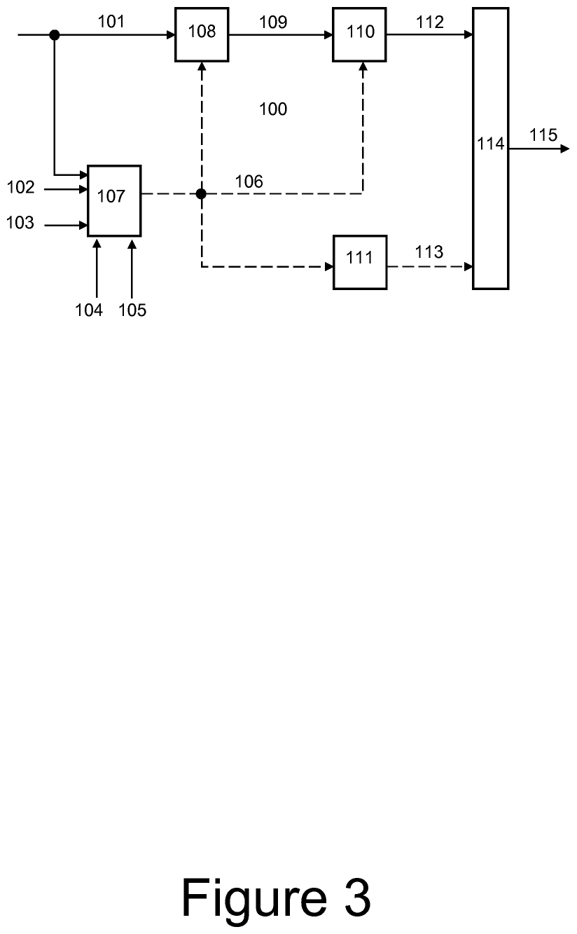

FIG. 3 possible structure of a first encoder;

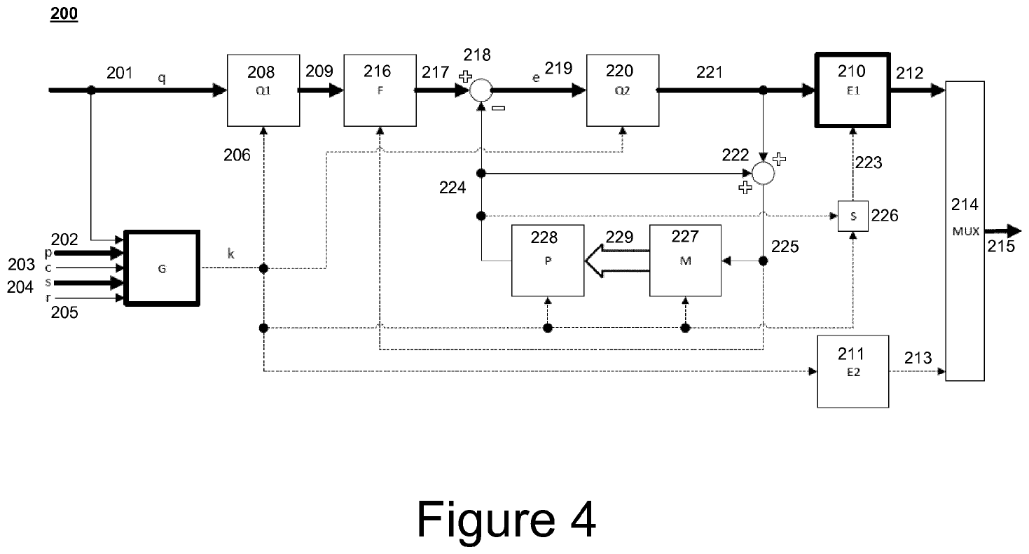

FIG. 4 possible structure of a second encoder.

A biological material sequence s=(s₁, . . . , s₁, . . . , s_{Ls}) is a sequence of length Ls, where the symbols s come from an alphabet S with cardinality /S/. A continuous fragment f, of length Lf, read out by a sequencing method from a sequence s can be written as f=(f₁, . . . , f_{ø}, . . . , f_{Lf}) A quality value q_{ϕ} may be associated with a symbol f_{ϕ}. Usually, there are corresponding quality values q_{ϕ}for all symbols f_{ϕ}.

Furthermore, since the sequencing process might be error-prone, the fragments may contain insertions and/or deletions of arbitrary size, with respect to the corresponding sequence s, or an arbitrary number of consecutive symbols that have not been read out correctly. Single symbol errors are in a biological context also known as single nucleotide polymorphisms or SNPs.

Generally, multiple fragments f⁽ⁱ⁾ might overlap due to redundant reading of the donor sequence s by the employed sequencing method. The number of times a specific locus I in a donor sequence s has been read out is called sequencing depth N at locus I. The average sequencing depth over all loci is called coverage.

Furthermore, multiple so-called homologous sequences sₕ containing symbols from the same alphabet S may exist or be assumed, where a specific fragment usually has been read out from one specific donor sequence. These sequences are in principle similar, but may contain arbitrary alterations. The total number of homologous donor sequences is referred to as the polyploidy h, which is a property of the organism being investigated.

If at a specific locus I all homologous sequences contain the same symbol, the sequences are denoted as being homozygous at that specific locus; otherwise, the sequences are denoted as being heterozygous at that specific locus. A specific symbol at a locus I on one sequence is called allele. Finally, a specific combination of symbols, i.e. alleles, across all h sequences at a locus I is called the genotype at locus I.

FIG. 1 shows a description of a first embodiment, whereby the data structure comprises a reference sequence s. Furthermore, the data structure includes in this example four continuous fragments f₁ to f₄, which are overlapped at a specific locus index I.

The reference sequence s and the continuous fragments f₁ to f₄ are nucleotide sequences, which comprises nucleotide symbols A, C, T and G.

All continuous fragments f₁ to f₄ are aligned to the reference sequence s. so that each position of a continuous fragment f₁ to f₄ corresponds to the correct position in the reference sequence s.

In the first step, for a specific locus I, the quality values of the corresponding symbols are determined. Shown in FIG. 1, at a specific locus I, there exists specific symbols in the continuous fragments f₁ to f₄. The first continuous fragment f₁ comprises at the specific locus I the symbol “A”. The second continuous fragment f₂ comprises also the symbol “A”. The third continuous fragment f₃ comprises at the specific locus I the symbol “C” and the fourth continuous fragment f₄ comprises at the specific locus I the symbol “T”.

A quality “q” is assigned to each a symbol at the specific locus I. The quality value is in the example in FIG. 1 determined as a number between 36 and 106.

The quality value 106 is assigned to the both symbols “A” of the first and the second continuous fragment f₁ and f₂. The symbols “C” and “T” of the third and fourth continuous fragment f₃ and f₄ are assigned to the quality value 36.

A high quality value means that the likelihood of correctness of the symbol in view of the reference sequence s is very high. A low quality value means a low likelihood correctness.

Therefore, in FIG. 1, the likelihood of correctness in regard to the symbol “A” is very high. The likelihood of correctness of the symbols “C” and “T” is very low.

Based on the determined quality values, an estimation certainty k is calculated. Based on the fact, those two identical symbols have a high quality value and another, different two symbols have a low quality value, a likelihood of correctness of each quality value in relation to the corresponding symbol is very high. Based on the quality values q, an estimation certainty k with a quality value of 100 is calculated. The estimation certainty k is calculated in form of a quality value derived from a quality value alphabet.

In the next step, each quality value q is transformed into a transformed quality value q′. Therefore, the determined quality values are transformed by setting each quality value to the estimation certainty if the estimation certainty is greater or equal than the quality value to be transformed. The quality value q₁ and q₂ corresponding to the symbols at the specific locus index I in the continuous fragment f₁ and f₂ are not transformed because these quality values are higher than the estimated certainty k. The other quality values q₃ and q₄ are set to the estimation certainty k, so that in the continuous fragments f₃ and f₄ at a specific locus I the symbols correspond to a quality value 100.

Based on these transforming of quality values, a better compression rate is realized by using well known compression algorithms.

On the second embodiment, based on the estimation certainty k, a quantization characteristic qc is selected. A quantization characteristic is a step function, which controls that a part of continuous quality values are assigned to one value. In FIG. 2, all quality values between 36 and 53 are assigned to a quantized quality value q′ of 50. The other quality values between 53 and 106 are quantized to a value of 100. That means, that the quality values according to the symbols “A” are set to 100 and the quality values to the symbols “C” and “T” are set to 50.

The method proposed herein has been designed, among others, to lossily compress and decompress the quality values by deriving what so far has been called the estimation certainty k for a locus I from the observable data. In the case of sequencing DNA material, the estimation certainty k is also called as a genotype certainty k. The genotype certainty k may than be used to control the working of one or more signal processing modules, such as filter modules and/or one or more coding modules, such as transform modules, quantization modules, prediction modules or entropy coders. The proposed method used statistics to control the acceptable (e.g. non-perceivable) loss.

Specifically, the genotype certainty of a locus might be used to control the quantization of quality values associated with that locus. More specifically, in one embodiment, the quantizer for said locus might be selected from a set of previously computed quantizers using the genotype certainty k.

As an alternative, a quantizer might be computed online using the genotype certainty. For example, the genotype distribution might be computed. The standard deviation of this distribution might be used to select or compute a quantizer for that locus. In an another embodiment the genotype certainty might be used to modify the quality values. For example, the frequencies of all observed symbols at said locus might be counted. If there is a high certainty regarding one specific symbol (i.e. one frequency is multiple times higher than other frequencies), the quality values supporting this symbol might be set to a high value (e.g. the highest value applicable).

Furthermore, the specific way of deriving the genotype certainty k may depend on the targeted application, such as e.g. haplotype calling in the case of genome sequencing, for the sequencing data or depending on general preferences of the user. Given the sequencing depth N at locus I, the immediately observable data are the symbols and the associated quality values of fragments f overlapping locus I. Furthermore, additional symbols in the vicinity of index I might be added to the observable data.

For the derivation of the genotype certainty k, either the complete observable data, or a subset of the observable data might be used.

Furthermore, a genotype certainty k as derived for one locus I might additionally be applied to other, e.g. neighboring loci.

The following text describes an embodiment of said method which has been designed to lossily compress quality values generated by a DNA sequencing machine.

FIG. 3 shows a first encoder structure. The encoder 100 gets as input quality values q 101, mapping positions p 102, CIGAR strings c 103, nucleotide sequences s 104, and the reference sequences r 105, as defined in the SAM file format specification.

The derivation of the genotype certainty k 106 is performed by module G 107 which gets as input the quality values q 101, the mapping positions p 102, the CIGAR strings c 103, the nucleotide sequences s 104, and the reference sequences r 105.

The genotype certainty k 106 may control the working of a quantization module Q1 108 which quantizes the quality values q 101 and outputs either quantizer indices 109 or representative quality values.

Examples of controlling the working of a quantization module include, but are not limited to, the following: The genotype certainty k 106 might be used to select a specific quantizer from a previously computed set of quantizers stored in module G 107. The quantizers could also be computed on-line instead of being selected from a previously computed set of quantizers. The quantizer indices 109 or representative quality values are then encoded by an entropy coder E1 110, which might also be controlled by the derived genotype certainty k 106, into a first bitstream 112.

Furthermore, the genotype certainty k 106 is encoded by entropy coder E2 111 into a second bitstream 113.

The entropy coder modules E1 110 and E2 111 might contain at least one entropy coding step such as e.g. run-length encoding, Huffman coding, Golomb coding, Rice coding, arithmetic coding, or a general-purpose coding, or a combination of these entropy coding methods. Furthermore, the entropy coders might be controlled by any number of statistical models (e.g. such as in CABAC). The outputs 112, 113 of both entropy coders E1 110 and E2 111 might then be multiplexed by the multiplexer module MUX 114 into a multiplexed bitstream 115 to be sent to a corresponding decoder.

In general, to form a coding system, the derivation of the genotype certainty k 106, carried out by module G107, might be combined with arbitrary signal processing techniques such as e.g. filtering or with arbitrary coding techniques such as e.g. transform coding, quantization, predictive coding, or general-purpose encodings. Any of said coding methods might be backwards-adaptive and/or controlled by the derived genotype certainty.

FIG. 4 shows a second, extended encoder as another example. The encoder shown in FIG. 4 comprises additional optional modules, but also some of the modules shown in FIG. 3 are optional in FIG. 4. Correspondingly to the first encoder structure of FIG. 3, here the encoder 200 gets as input the quality values q 201, the mapping positions p 202, the CIGAR strings c 203, the nucleotide sequences s 204, and the reference sequences r 205.

The derivation of the genotype certainty k 206 is here performed by module G 207 which gets as input the quality values q 201, the mapping positions p 202, the CIGAR strings c 203, the nucleotide sequences s 204, and the reference sequences r 205.

Again, the genotype certainty k 206 may control the working of a quantization module Q1 208 which quantizes the quality values q 201 and outputs either quantizer indices 209 or representative quality values.

Now, the quantizer indices 209 or representative quality values enter into a filter F 216. The module F 216 is a filter module to control quality value trends. These trends might be shared by multiple fragments.

For example, the employed sequencing technology might produce fragments with low-quality beginnings and/or endings which can be controlled, e.g. smoothed, by module F 216. The filter module F 216 might be backwards-adaptive, i.e. it might be controlled by already processed reconstructed quality values, as indicated by the optional control signal 225.

The optional module M 227 is a memory module, holding m already processed reconstructed quality values 225.

The memorized reconstructed quality values 229, or a subset of the reconstructed quality values, might be used to predict quality values with the optional prediction module P 228.

The memory module M 227 and/or the prediction module P 228 might be controlled by the derived genotype certainty k 206, as indicated by the corresponding optional control signals.

For example, the genotype certainty k 206 might control the number of memorized reconstructed quality values passed to the prediction module P 228.

The optional module Q2 220 is a quantization module for the quantization of the prediction errors e 219. Module Q2 220 might also be controlled by the derived genotype certainty k 206 in the same fashion as module Q1 208.

The entropy coder module E1 210 might also be controlled by the genotype certainty k 206 and/or the predicted quality values 224.

Furthermore, it is possible to add additional coding modules to the encoder structure shown in FIG. 4. For example, an additional memory module and an additional prediction module might be added to predict the genotype certainties k 206, before they are passed to module E2 211.

As another example, the genotype certainty k 206 might not be transmitted to the decoder, as indicated by the module E2 211 being marked as optional, if the quality values q are not used to derive the genotype certainty k, with other words if the genotype certainty k 206 is derived from nothing more than the mapping positions p 202, the CIGAR strings c 203, the nucleotide sequences s 204, and the reference sequences r 205, or any subset thereof.

The decoder would then as well be able to decode the encoded quality values 212, because the signals forming the input to module G 207 can be transmitted as side information to the decoder.

Examples for the Derivation of the Genotype Certainty This section describes an example embodiment of the module G 107, 207 as shown in FIG. 3 and FIG. 4. For this embodiment, we assume that quality values are generated during the sequencing of biological, e.g. DNA, material.

More specifically, the example embodiment is designed to lossily compress quality values produced by the sequencing of a donor genome or at least one part of a donor genome with polyploidy h and thus h homologous chromosomes.

At any locus I in the donor genome, the genotype is represented by a random variable gt drawn from a genotype alphabet GT with cardinality /GT/. The genotype gt is the set of alleles found at a locus I across all donor sequences, i.e. all chromosomes, where more than h donor sequences might be assumed.

The genotypes are a set of alleles:

gt=(A₁, . . . , A_{α}, . . . , Aₕ)

where the alleles A_{α}are individually drawn from the allele alphabet A with cardinality /A/, which here is the same as the alphabet S. The number of possible genotypes |GT| can be derived by computing all possible allele combinations with repetitions.

Thus,

$$\vert GT \vert = ( \begin{matrix}\vert S \vert + h - 1 \\ \vert S \vert - 1\end{matrix} )$$

For example, in the case of DNA sequencing the allele alphabet A={A, C, G, T}consists of |A|=4 symbols. As a sidenote, an additional symbol “N” may be emitted by sequencing machines if no decision about a nucleotide at a specific position could be made. However, as real DNA sequences cannot contain “N”, we omit it here.

For a diploid organism with h=2, the above formula results in (5/3)=10possible genotypes. In enumeration, they are {AA, AC, AG, AT, CC, CG, CT, GG, GT, TT}.

A set of reads is assumed that are aligned to a reference sequence (i.e. genome) or that have been aligned by a de-novo assembler. It is further assumed that the reads have been sorted by their mapping positions.

Given such set of reads, let us denote by N the number of reads covering locus I, i.e. the sequencing depth at locus I. Let Yᵢ be the symbol from read i covering the locus I and Qᵢthe value of the corresponding quality value.

The goal is now to compute the posterior distribution of the genotype gt, given the observable data

(Y, Q)={(Yᵢ, Qᵢ)}ᵢ₌₁^{N}.

The posterior probability is proportional to the likelihood times the prior:

P(gt|(Y, Q)) ∝P ((Y, Q)|gt)·P(gt)

The likelihood is given by

P((Y, Q)|gt)=Πᵢ₌₁^{N} P((Yᵢ,Qᵢ)|gt),

where P((Yᵢ,Qᵢ)|gt) is the likelihood of having observed (Yᵢ, Qi) given that the genotype was gt.

Recall that the genotype gt is expressed as gt=(A₁, . . . , A_{α}, . . . , Aₕ) where A_{α} is the α-th allele. Thus, according to the principle of indifference, the likelihood is given by

$$P ( ( {Y}_{i} , {Q}_{i} ) \vert gt = ( {a}_{1} , … , {a}_{h} ) ) = {\sum }_{α = 1}^{h} R with$$

$$R = \frac{P ( ( {Y}_{i} , {Q}_{i} ) \vert {A}_{α} = {a}_{α} )}{h}$$

where P((Yᵢ, Qᵢ)|A_{α}=α_{α}) shall denote the likelihood of having observed (Yᵢ, Qᵢ) given the assumption that the true symbol was the alleleα_{α}.

This probability is given by

$$P ( ( {Y}_{i} , {Q}_{i} ) \vert {A}_{α} = {a}_{α} ) = \{ \begin{matrix}1 - {10}^{- {Q}_{i} / 10} , & {Y}_{i} = {a}_{α} \\ \frac{{10}^{- {Q}_{i} / 10}}{\vert A \vert - 1} , & {Y}_{i} \neq {a}_{α}\end{matrix}$$

Given the posterior distribution P(gt|(Y, Q)) of the genotype gt, we calculate a metric M(P(gt|(Y, Q)))over the distribution. Any metric such as e.g. the entropy, the Kullback-Leibler divergence, or the difference of the largest likelihood minus the second largest likelihood might be applied.

For example, a high entropy can be interpreted as a high uncertainty regarding the genotype given the observable data and vice versa.

The metric M is used to derive the genotype certainty k as

k←f (M(P(gt|(Y, Q)))),

where f might be any monotonously non-decreasing function. The function f might for instance be a quantization function mapping the possible metric values to an integer set of possible genotype certainties k.

Specifically, as one example configuration, the function f may be one that has 3 possible output values, a first output value that is yielded for metric values in a lower range, a second output value that is yielded for metric values in a middle range, and a third output value that is yielded for metric values in an upper range. Having such three distinct output values of the genotype certainty k, allows one to control a quantization in such a way that quality values associated to symbols aligned to loci of high estimation certainty are quantized more coarsely than quality values associated to symbols aligned to loci of low estimation certainty, namely by selecting for instance a four-step quantizer whenever the third output value has been yielded, selecting an eight step quantizer whenever the second output value has been yielded, and leaving the quality values as they are whenever the first output value has been yielded.

As an alternative, any of the mentioned signals might be used to estimate the genotype certainty k for the given locus I.

For example, the entropy H(P((Y, Q)|gt)) might as well be used as the metric M. Finally, the derived genotype certainty k at locus I will be used to select a quantizer which is applied on all quality valuesQᵢcovering locus I.

1. A method for encoding of quality values of a data structure, whereby said data structure includes a plurality of continuous fragments, each continuous fragment comprises a sequence of symbols derived from a symbol alphabet and corresponds to a segment of one reference sequence of one or more reference sequences, whereby each continuous fragment is aligned to locus indexes of one of said reference sequence and at least a portion of said continuous fragments overlap at an aligned locus index, and includes, further, a plurality of quality values, each quality value is derived from a quality value alphabet and is assigned to a corresponding symbol of one of the continuous fragments, whereby each quality value indicates a likelihood that the corresponding symbol in the corresponding continuous fragment is correct, wherein the method comprises the steps executable by a data processing system:
determining the quality values at a specific locus index, which are assigned to symbols of continuous fragments aligned to said specific locus index;
calculating an estimation certainty at the specific locus index based on the determined quality values, whereby said estimation certainty indicates a likelihood of correctness for each quality value of the determined quality values in relation to the corresponding symbols; and
encoding the determined quality values by transform of each determined quality values into a transformed quality value based on the calculated estimation certainty.

2. The method according to claim 1, wherein the estimation certainty is calculated in a form of a quality value derived from the quality value alphabet and the determined quality values are transformed by setting each quality value to the estimation certainty if the estimation certainty is greater or equal than the quality value to be transformed.

3. The method according to claim 1, further comprising compressing the transformed quality values using a compression algorithm.

4. The method according to claim 1, further comprising selecting a quantization characteristic based on the estimation certainty at the specific locus index, said quantization characteristic associates all quality values of the quality value alphabet to one or more quantized quality values, whereby the determined quality values are transformed by quantizing each determined quality value into a quantized quality value based on said selected quantization characteristic, whereby the estimation certainty or a quantization characteristic identifier related to the selected quantization characteristic is assigned to the specific locus index and the quantized quality values are used as transformed quality values.

5. The method according to claim 4, wherein the quantization characteristic is selected based on the estimation certainty in such a way that determined quality values at a first locus index with a first estimation certainty are quantized more coarsely than the determined quality values at a second locus index with a second estimation certainty, if the first estimation certainty is higher than the second estimation certainty.

6. The method according to claim 4, further comprising selecting a step size of the quantization characteristic is based on the estimation certainty.

7. The method according to claim 4, wherein an entropy coding step of the quantized quality values is controlled by using the estimation certainty.

8. The method according to claim 1, wherein each continuous fragment corresponds to a segment of one reference sequence of two or more reference sequences, whereby
all possible symbol combinations are determined at the specific locus index based on the total number of corresponding reference sequences,
for each symbol combination, a likelihood of occurrence is calculated based on the determined quality values, and
the estimation certainty at the specific locus index is calculated based on the likelihood of occurrence of each symbol combination.

9. The method according to claim 8, wherein the method steps are performed for each locus index of a corresponding reference sequence.

10. The method according to claim 9, wherein the corresponding reference sequence is a donor genome sequence of a plurality of nucleotides, whereby the symbol alphabet includes at least the four different nucleotides, the continuous fragment is a readout, whereby the readout is a partial sequence of plurality of nucleotides, and the quality value express the confidence that the corresponding nucleotide has been read out correctly.

11. The method according to claim 1, wherein said data structure includes one or more reference sequences.

12. A method for decoding of transformed quality values of a data structure, the transformed quality values are encoded by a method of encoding of quality values according to claim 4, wherein the method comprises the steps executable by a data processing system:
determining the transformed quality values at a specific locus index;
determining the estimation certainty or the quantization characteristics identifier assigned to the specific locus index;
selecting a quantization characteristic based on the determined estimation certainty or quantization characteristic identifier; and
decoding the determined transformed quality values by retransform of each determined transformed quality value into a re-quantized quality value based on the selected quantization characteristic.

13. A computer program on a non-transitory computer readable medium arranged to execute the encoding method according to claim 1.

14. A hardware device arranged to execute the encoding method according to claim 1.

15. A computer program on a non-transitory computer readable medium arranged to execute the decoding method according to the claim 12.

16. A hardware device arranged to execute the decoding method according to the claim 12.
